(19) United States
(12) Patent Application Publication
(10) Pub. No.: US 2022/0265985 A1
(43) Pub. Date: Aug. 25, 2022

(54) LUER LOCK INTRAVENOUS CONNECTOR

(71) Applicant: CareFusion 303, Inc., San Diego, CA (US)

(72) Inventor: Mark Steven THOMAS, Phoenix, AZ (US)

(21) Appl. No.: 17/588,668

(22) Filed: Jan. 31, 2022

Related U.S. Application Data
(60) Provisional application No. 63/153,281, filed on Feb. 24, 2021.

(51) Int. Cl. A61M 39/16; A61M 5/162

(52) U.S. Cl. CPC A61M 39/16; A61M 5/162; A61M 2205/586; A61M 2205/02

(57) ABSTRACT

Luer lock connectors are provided that include an inner core of a hard material, the inner core sized and shaped to mate with an inner portion of an opposing luer lock connector, an outer layer of a soft material disposed on an outer surface of the inner core, and a plurality of grip features disposed on an outer surface of the outer layer, wherein the plurality of grip features are configured to provide a slip resistant grip surface. Infusion sets and infusion set luer lock connectors are also provided.

### CROSS-REFERENCE TO RELATED APPLICATION

This application claims the benefit of priority under 35 U.S.C. § 119 to U.S. Provisional Patent Application Serial No. 63/153,281, entitled “LUER LOCK INTRAVENOUS CONNECTOR,” filed on Feb. 24, 2021, the entirety of which is incorporated herein by reference.

### TECHNICAL FIELD

The present disclosure generally relates to infusion set intravenous (IV) connectors, in particular luer lock IV connectors.

### BACKGROUND

Typical infusion or intravenous (IV) sets are constructed by joining polymeric tubing segments to polymeric components, many of which use luer lock connectors. These IV sets are then used with infusion pumps or gravity systems to provide fluids to a user, such as a patient. However, typical luer lock connectors are made of hard plastic that is slippery when the connector is wet or when a user's hand is wet, thus making it difficult to grip and undo the connector when changing out an IV component or IV set. In addition, the hardness of the typical connectors can cause skin tears and pressure sores when the connectors are pressed against a patient's skin for a prolonged period of time.

In order to deal with this issue, some typical solutions are to use pliers or forceps to forcibly grip the connector in order to disconnect or connect IV components or IV sets. However, this solution can damage the connector and requires the user to have pliers or forceps on hand. In addition, the large amount of force required to disconnect the connector may cause the catheter to pull or rip out of the patient. Other solutions have been to wrap the connector in cotton gauze and tape to help create come type of padding between the connector and the patient's skin. This solution requires additional time from the user to connect and prepare IV components or IV sets, as well as an increased cost for the additional gauze and tape materials for every connector.

For these reasons, it is desirable to provide luer lock connectors for use with IV components and IV sets that are easy to grip and cushioned.

### SUMMARY

In one or more embodiments, a luer lock connector includes an inner core comprising a hard material, the inner core sized and shaped to mate with an inner portion of an opposing luer lock connector, an outer layer disposed on an outer surface of the inner core, the outer layer comprising a soft material, and a plurality of grip features disposed on an outer surface of the outer layer, wherein the plurality of grip features are configured to provide a slip resistant grip surface.

In one or more embodiments, an infusion set includes an infusion set component and a luer lock connector. The luer lock connector includes an inner core formed from a hard material comprising at least one of polycarbonate, nylon and polyethylene, an outer layer disposed on an outer surface of the inner core, the outer layer formed from a soft material comprising at least one of silicone, polyurethane and latex free silicone rubber, and a plurality of grip features disposed on the outer layer, the plurality of grip features nfigured to provide a slip resistant grip surface.

In one or more embodiments, an infusion set luer lock connector includes an inner core formed from a hard material comprising at least one of polycarbonate, nylon and polyethylene, the inner core sized and shaped to mate with a receiving portion of an opposed gender luer lock connector, an outer layer disposed on an outer surface of the inner core, the outer layer formed from a soft material comprising at least one of silicone, polyurethane and latex free silicone rubber, and a plurality of grip features disposed on the outer layer, the plurality of grip features comprising at least one of a plurality of raised features and a plurality of recessed features, wherein the grip features are configured to provide a slip resistant grip surface.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIGS. 2A-2C are top views of a typical assembled IV infusion set and typical IV extension lines.

FIG. 3 depicts a front view of a luer lock IV connector, according to aspects of the disclosure.

FIG. 4 depicts a front view of an outer layer of a luer lock IV connector, according to aspects of the disclosure.

FIG. 5 depicts a front view of an outer layer of a luer lock IV connector, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

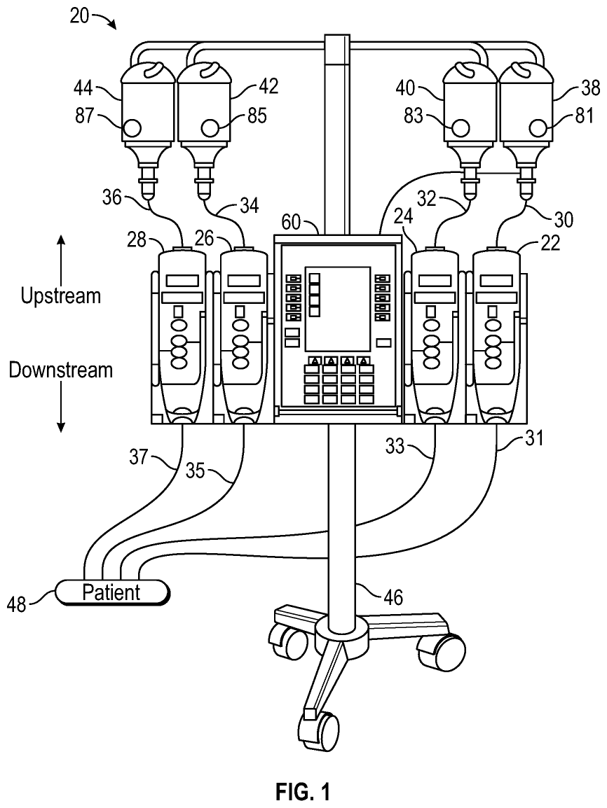

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

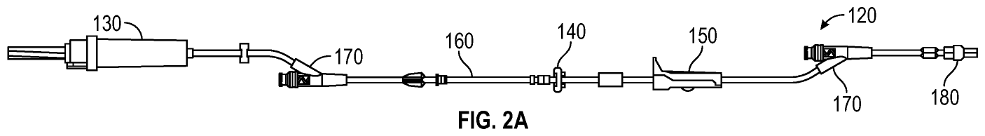

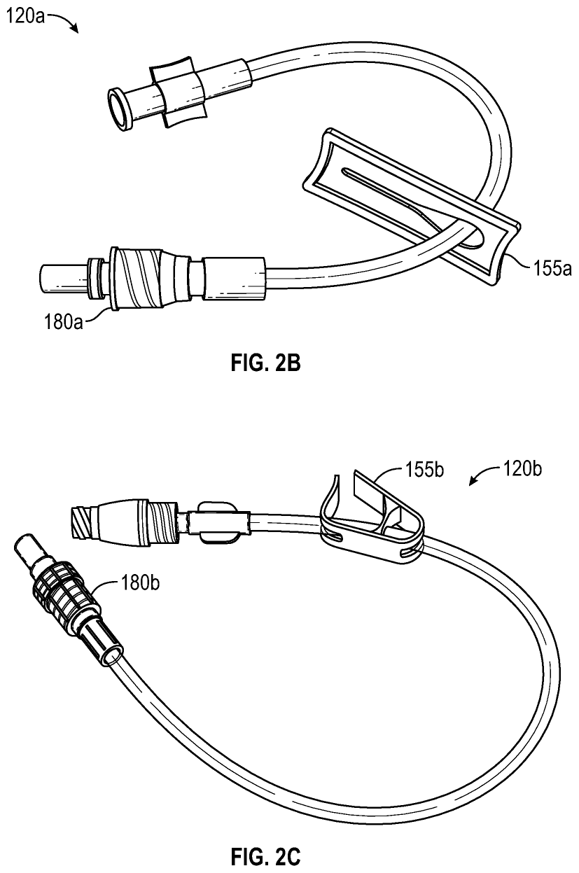

Typically, medical fluid administration sets have more parts than are shown in FIG. 1, such as those shown in FIGS. 2A-2C. Infusion sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIGS. 2A, an infusion set 120 may include a drip chamber 130, a check valve 140 and a roller clamp 150 connected together by tubing 160. The infusion set 120 may also include a Y-site 170 having a Y-shaped junction with a needleless connector 175, as well as a luer lock connector 180 at the end of the infusion set 120. The luer lock connector 180 may be used for connection to a catheter inserted into a patient, for example. The infusion set 120 may include additional infusion components and may be formed of any combination of components and the tubing 160.

As shown in FIG. 2B, an IV extension set 120a may be used to connect any two infusion components or devices, such as IV sets, infusion pumps, syringe pumps and the like. IV extension set 120a includes a luer lock connector 180a and a flow controller 155a. Similarly, as shown in FIG. 2C, another IV extension set 120b includes a luer lock connector 180b and a flow controller 155b.

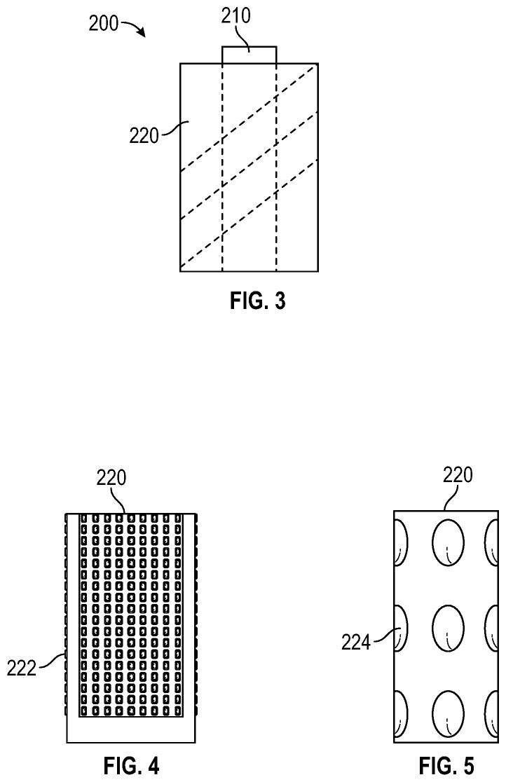

As shown in FIG. 3, a luer lock connector 200 is provided according to aspects of the disclosure. Luer lock connector 200 has an inner core 210 formed from a hard material, such as polycarbonate, nylon, polyethylene, and the like. The inner core 210 may mate with a standard receptacle of an infusion set device or component (e.g., needle free port, catheter set, pump port) that is configured to connect with a luer lock connector.

An outer layer 220 is disposed around the inner core 210, the outer layer 220 being formed of a material that is soft and cushioned. For example, the outer layer 220 may be formed of any suitable material, such as silicone, polyurethane, a latex free silicone rubber, and the like, or any combination thereof. The soft cushioning property of the outer layer 220 allows the luer connector 200 to be pressed against a patient's skin for a prolonged period of time without causing skin tears or pressure sores.

The outer layer 220 may be latex free, providing for use in a wide range of medical applications, including those that proscribe latex contact with the patient's skin (e.g., patient has latex allergy). The outer layer 220 may be slip resistant when wet and may be textured and/or contoured to provide for an improved grip when the luer lock connector 200 is to be loosened or tightened. The outer layer 220 may be formed to hold up to gas sterilization and gamma sterilization.

As shown in FIG. 4, outer layer 220 may include raised features 222, such as the bumps as shown, which may provide texturing for non-slip and/or easy grip properties. The raised features 222 may be any suitable feature, such as ridges or other raised areas, for example. Similarly, as shown in FIG. 5, outer layer 220 may include recessed features 224, such as the indentations shown, which may provide texturing for non-slip and/or easy grip properties. The recessed features 224 may be any suitable feature, such as channels or valleys, for example. Outer layer 220 may include any combination of raised features 222, recessed features 224 and any other suitable features that provide a non-slip or low slip grip, as well as an easy and comfortable grip.

In aspects of the disclosure, luer lock connector 200 may be formed in any suitable manner. For example, an outer layer material may be overmolded onto a standard luer lock connector (e.g., connector 180, 180a, 180b) to form a luer lock connector 200 with a soft, non-slip and easy gripping outer layer 220. Here, the luer lock connector 200 may be a larger size and or shape than the standard luer lock connector.

As another example, a standard luer lock connector (e.g., connector 180, 180a, 180b) may be trimmed, peeled, shaved or any other manner of removing an outer portion of the standard luer lock connector body, and then a new outer layer material may be overmolded onto the trimmed standard luer lock connector to form a luer lock connector 200 with a soft, non-slip and easy gripping outer layer 220. The outer layer 220 may be sized to equal the removed outer portion of the standard luer lock connector so that the newly formed luer lock connector 200 has the same size and shape as the original standard luer lock connector.

As yet another example, an outer layer material may be overmolded onto an inner core 210 to form a luer lock connector 200 with a soft, non-slip and easy gripping outer layer 220.

The inner core 210 may be sized and shaped to mate with the same standard receptacles as does the inner portion of a standard luer lock connector, while the outer layer 220 may be sized so that the luer lock connector 200 has the same size and shape as the original standard luer lock connector. In aspects of the disclosure, the inner core 210 and the outer layer 220 may be sized and shaped to provide a luer lock connector 200 with a smaller footprint and/or lower profile than a standard luer lock connector.

In one or more embodiments, a luer lock connector comprises an inner core comprising a hard material, the inner core sized and shaped to mate with an inner portion of an opposing luer lock connector, an outer layer disposed on an outer surface of the inner core, the outer layer comprising a soft material, and a plurality of grip features disposed on an outer surface of the outer layer, wherein the plurality of grip features are configured to provide a slip resistant grip surface.

In aspects of the disclosure, the inner core is formed from a hard material comprising at least one of polycarbonate, nylon and polyethylene. In aspects of the disclosure, the inner core comprises a male mating portion configured to sealingly mate with a female portion of the opposing luer lock connector. In aspects of the disclosure, the inner core comprises a female mating portion configured to sealingly mate with a male portion of the opposing luer lock connector. In aspects of the disclosure, the outer layer is formed from a soft material comprising at least one of silicone, polyurethane and latex free silicone rubber. In aspects of the disclosure, the outer layer is latex free. In aspects of the disclosure, the outer layer is configured to withstand gas sterilization. In aspects of the disclosure, the outer layer is configured to withstand gamma ray sterilization.

In aspects of the disclosure, the plurality of grip features comprises raised features. In aspects of the disclosure, the raised features comprise bumps. In aspects of the disclosure, the raised features comprise ridges. In aspects of the disclosure, the plurality of grip features comprises recessed features. In aspects of the disclosure, the recessed features comprise indentations. In aspects of the disclosure, the recessed features comprise channels. In aspects of the disclosure, the plurality of grip features comprises a combination of raised features and recessed features. In aspects of the disclosure, the outer layer is overmolded onto the inner core.

In one or more embodiments, an infusion set comprises an infusion set component and a luer lock connector. The luer lock connector comprises an inner core formed from a hard material comprising at least one of polycarbonate, nylon and polyethylene, an outer layer disposed on an outer surface of the inner core, the outer layer formed from a soft material comprising at least one of silicone, polyurethane and latex free silicone rubber, and a plurality of grip features disposed on the outer layer, the plurality of grip features nfigured to provide a slip resistant grip surface.

In aspects of the disclosure, the outer layer is latex free and is configured to withstand gas sterilization and gamma ray sterilization. In aspects of the disclosure, the grip features comprise one of raised features and recessed features.

In one or more embodiments, an infusion set luer lock connector comprises an inner core formed from a hard material comprising at least one of polycarbonate, nylon and polyethylene, the inner core sized and shaped to mate with a receiving portion of an opposed gender luer lock connector, an outer layer disposed on an outer surface of the inner core, the outer layer formed from a soft material comprising at least one of silicone, polyurethane and latex free silicone rubber, and a plurality of grip features disposed on the outer layer, the plurality of grip features comprising at least one of a plurality of raised features and a plurality of recessed features, wherein the grip features are configured to provide a slip resistant grip surface.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A luer lock connector, comprising:
an inner core comprising a hard material, the inner core sized and shaped to mate with an inner portion of an opposing luer lock connector;
an outer layer disposed on an outer surface of the inner core, the outer layer comprising a soft material; and
a plurality of grip features disposed on an outer surface of the outer layer, wherein the plurality of grip features are configured to provide a slip resistant grip surface.

2. The luer lock connector of claim 1, wherein the inner core is formed from a hard material comprising at least one of polycarbonate, nylon and polyethylene.

3. The luer lock connector of claim 1, wherein the inner core comprises a male mating portion configured to sealingly mate with a female portion of the opposing luer lock connector.

4. The luer lock connector of claim 1, wherein the inner core comprises a female mating portion configured to sealingly mate with a male portion of the opposing luer lock connector.

5. The luer lock connector of claim 1, wherein the outer layer is formed from a soft material comprising at least one of silicone, polyurethane and latex free silicone rubber.

6. The luer lock connector of claim 1, wherein the outer layer is latex free.

7. The luer lock connector of claim 1, wherein the outer layer is configured to withstand gas sterilization.

8. The luer lock connector of claim 1, wherein the outer layer is configured to withstand gamma ray sterilization.

9. The luer lock connector of claim 1, wherein the plurality of grip features comprises raised features.

10. The luer lock connector of claim 9, wherein the raised features comprise bumps.

11. The luer lock connector of claim 9, wherein the raised features comprise ridges.

12. The luer lock connector of claim 1, wherein the plurality of grip features comprises recessed features.

13. The luer lock connector of claim 12, wherein the recessed features comprise indentations.

14. The luer lock connector of claim 12, wherein the recessed features comprise channels.

15. The luer lock connector of claim 1, wherein the plurality of grip features comprises a combination of raised features and recessed features.

16. The luer lock connector of claim 1, wherein the outer layer is overmolded onto the inner core.

17. An infusion set, comprising:
an infusion set component; and
a luer lock connector, the luer lock connector comprising:
an inner core formed from a hard material comprising at least one of polycarbonate, nylon and polyethylene;
an outer layer disposed on an outer surface of the inner core, the outer layer formed from a soft material comprising at least one of silicone, polyurethane and latex free silicone rubber; and
a plurality of grip features disposed on the outer layer, the plurality of grip features configured to provide a slip resistant grip surface.

18. The infusion set of claim 17, wherein the outer layer is latex free and is configured to withstand gas sterilization and gamma ray sterilization.

19. The infusion set of claim 17, wherein the grip features comprise one of raised features and recessed features.

20. An infusion set luer lock connector, comprising:
an inner core formed from a hard material comprising at least one of polycarbonate, nylon and polyethylene, the inner core sized and shaped to mate with a receiving portion of an opposed gender luer lock connector;
an outer layer disposed on an outer surface of the inner core, the outer layer formed from a soft material comprising at least one of silicone, polyurethane and latex free silicone rubber; and
a plurality of grip features disposed on the outer layer, the plurality of grip features comprising at least one of a plurality of raised features and a plurality of recessed features, wherein the grip features are configured to provide a slip resistant grip surface.
